(19) United States
(12) Patent Application Publication
(10) Pub. No.: US 2022/0105331 A1
(43) Pub. Date: Apr. 7, 2022

(54) Periodic Delivery and Release System for Hormone Pellets

(71) Applicant: Odilza Vital Fill, (US)

(72) Inventors: Odilza Vital Fill, Lawrence, NY (US); Mitchell Kurk, Lawrence, NY (US)

(21) Appl. No.: 17/495,580

(22) Filed: Oct. 6, 2021

Related U.S. Application Data
(60) Provisional application No. 63/088,098, filed on Oct. 6, 2020.

(51) Int. Cl. A61M 37/00

(52) U.S. Cl. CPC A61M 37/0069; A61M 2205/3303; A61M 2205/18

(57) ABSTRACT

The periodic delivery and release system is a system for treating a medical condition through pellet therapy. The system aims to improve upon pellet therapy by releasing hormones periodically, such that the pellets may be used to treat hormonal issues for the female body and treat diseases like prostrate cancer in men through the cyclic release of hormones. The system includes different methods of application, as well as a delivery algorithm, which provides the patient with an exact cycle of hormone delivery, followed by a temporary pause of hormone delivery. Further, the system employs biodegradable components that are bioidentical to the hormones produced by the human body. Thus, the system releases a constant dose of hormones with intervals into the body, avoiding the negative effects of the high levels of hormones for an extended period of time and fast rate drop of hormones.

The current application claims a priority to the U.S. Provisional Patent application Ser. No. 63/088,098 filed on Oct. 6, 2020.

### FIELD OF THE INVENTION

The present invention generally relates to a delivery system for hormone pellets. More specifically, the present invention is a hormone pellet delivery system for treating a medical condition using a periodic release algorithm.

### BACKGROUND OF THE INVENTION

Pellet therapy has been studied over the span of 80 years and has proven to be safe and effective, while also data has proven to prevent cardiovascular diseases, breast cancer relapse, osteoporosis, dementia, and various other maladies. Although, this method has proven to be successful, there is always a tendency to strive for safer and more effective methods and treatments. Other alternatives include hormonal creams and pills, which lack the effectiveness of pellet therapy. Other methods, such as oral and transdermal therapy, result in high levels of hormones all at once without the interval that allowed female tissue to evolute to apoptosis. After the high level input of hormones, the level remains too high for months and then, the levels tend to drop off at a fast rate obligating a new procedure every 90 or 120 days. The outcome of a fast rate drop of hormones results in extreme mood and energy fluctuation over time and a new invasive procedure must be performed. Various methods of insertion are currently practiced for pellet therapy, one of the most common methods is in an in-office procedure. Typically, hormone pellet therapy uses hormones extracted from natural plants with the same chemical structure produced in human body, in order to replicate the body's normal hormone level.

An objective of the present invention to better improve upon pellet therapy that release hormones periodically, such that the pellets may be used to treat hormonal issues for the female body and treat diseases like prostrate cancer in men through the cyclic release of hormones. The present invention comprises different methods of application for pellet therapy, as well as a delivery algorithm, which provides the patient with an exact cycle of hormone delivery, and then a temporary pause of hormone delivery, from which after the delivery algorithm re-activates the hormone delivery. Further, the present invention employs biodegradable components that are bioidentical to the hormones produced by the human body. The following document aims to provide a clear and concise description of the system that implements a method for the present invention.

### BRIEF DESCRIPTION OF THE DRAWINGS

FIG. 1 is a block diagram showing a preferred embodiment of a medication release mechanism.

FIG. 2 is a flowchart illustrating the overall method for employing the present invention.

FIG. 3 is a flowchart illustrating an example method of functioning of the present invention, wherein the pellet is catered for a female human body.

FIG. 4 is a flowchart illustrating a method of operation of a monitoring apparatus.

FIG. 5 is a schematic view of the present invention, wherein the medication release mechanism comprises a plurality of membrane layers and medication layers.

FIG. 6 is a flowchart illustrating a method of the present invention.

FIG. 7 is a perspective view of a pellet for the present invention.

### DETAIL DESCRIPTIONS OF THE INVENTION

All illustrations of the drawings are for the purpose of describing selected versions of the present invention and are not intended to limit the scope of the present invention.

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure includes many aspects and features. One or more steps of the method may be automatically initiated, maintained, and/or terminated based on one or more predefined conditions.

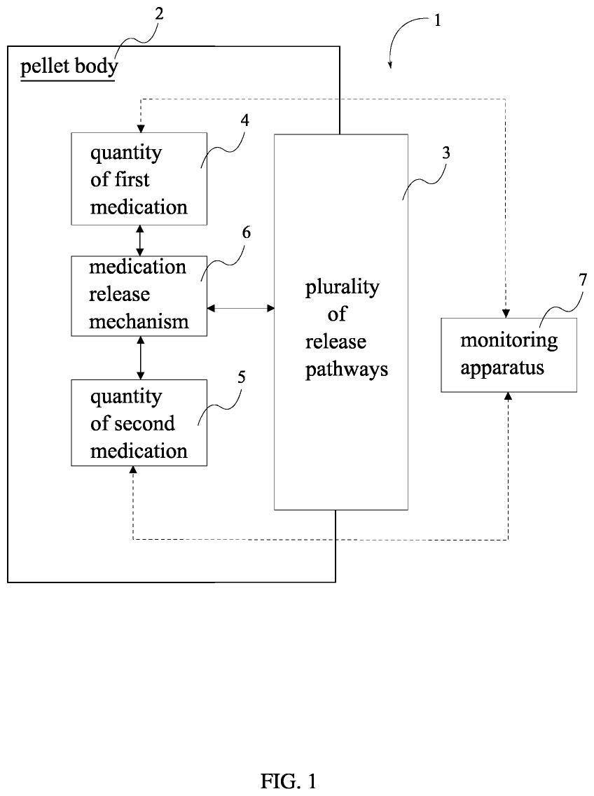

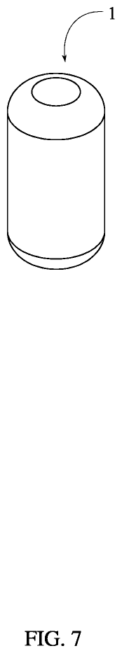

In reference to FIG. 1 through FIG. 7, the present invention is a pellet 1. According to a preferred embodiment of the present invention, the pellet 1 comprises a pellet body 2, a plurality of release pathways 3, a quantity of a first medication 4, a quantity of a second medication 5, and a medication release mechanism 6. Preferably, the pellet 1 is a hormone pellet, wherein the size of the hormone pellet varies depending on the dosage of hormones. However, for the preferred embodiment the pellet 1 comprises a diameter from three to four millimeters by one to two millimeters. Additionally, a variance in size does not limit the scope of the invention, as the crucial factor of the invention is the method and release algorithm rather than the shape and dimension of the hormone pellet.

It is an aim of the present invention to solve hormonal imbalances with a cyclic release algorithm. In other words, in the preferred embodiment, the present invention aims to dispense a particular hormone for a first period of time followed by a pause in the release of that hormone for a second period of time, like the hormone release cycle in a female body. To accomplish this, the plurality of release pathways 3 is integrated into the pellet body 2. Preferably, the medication release mechanism 6 and the plurality of release pathways 3 relates to a technique and method of releasing hormones stored within the pellet into the user's body, such as through layers of medication that dissolves gradually, dispensing holes etc. Further, release pathways 3 and release mechanism 6 could also refer to mechanical devices like a pump and holes. Alternatively, the medication release mechanism 6 could refer to chemical reactions, wherein the dispenser is an ion pump, and the release pathways 3 are ion gates that prevent medication release if a certain ion potential is not reached. More specifically, the present invention may comprise two different pathways and release mechanism catering to two different medications. To accomplish this, the quantity of the first medication 4, the quantity of the second medication 5, and the medication release mechanism 6 are housed within the pellet body 2. In the preferred embodiment, the medication release mechanism 6 is operatively coupled in between the plurality of release pathways 3, the quantity of the first medication 4 and the quantity of the second medication 5, wherein the medication release mechanism 6 dispenses a portion of the quantity of the first medication 5 and a portion of the quantity of the second medication 6. Preferably, the quantity of the first medication 4 may be a hormone that induces a chemical effect on the user's body, and the quantity of the second medication 5 may be a sterol, a vitamin, or a placebo medication that does not induce any chemical effect on the user's body.

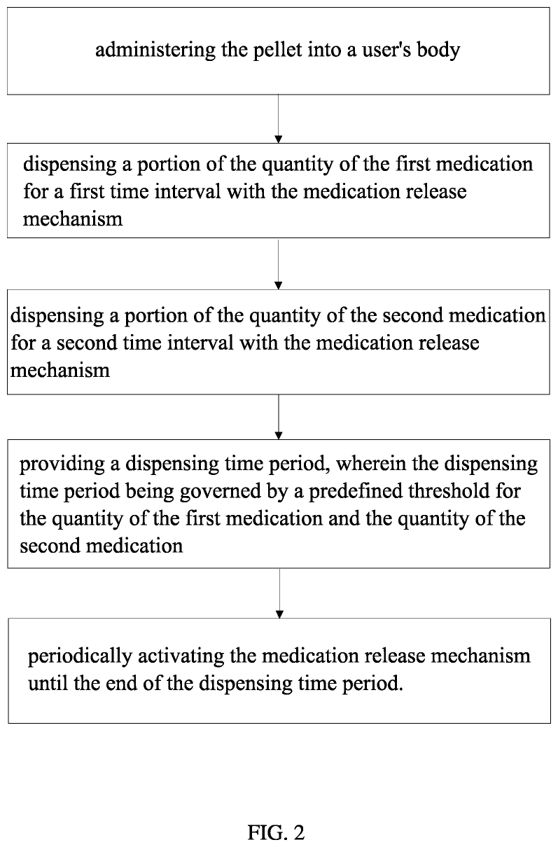

In reference to FIG. 2, to achieve the above-described functionality, the present invention comprises the following method of execution. Firstly, the method begins at administering the pellet into a user's body. Preferably, the method for insertion of the hormone pellet is via a subcutaneous injection. In other words, the method for insertion of the hormone pellet is via an injection which penetrates under the skin to subcutaneous tissue and then the hormone pellet is released. Additionally, the pellet 1 is easily localized by a special markup and may be localized by a simple ultrasound and removed from a patient's body if the treatment needs to be interrupted. Following insertion, the method comprises dispensing the portion of the quantity of the first medication 4 for a first time interval with the medication release mechanism 6, followed by dispensing the portion of the quantity of the second medication 5 for a second time interval with the medication release mechanism.

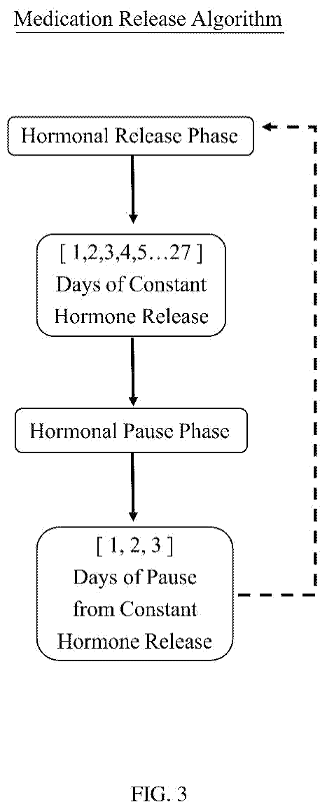

For example, in reference to FIG. 3, when the pellet 1 is catered for a female body, the medication release algorithm delivers a constant dose of hormones to the female body for twenty seven consecutive days. During this time, the pellet 1 delivers the hormone dose as it naturally would without any interruption. Thus, the portion of the quantity of the first medication 4 is a hormone that mimics the female hormone and the first time interval is twenty seven days. After, twenty seven days the pellet 1 pauses hormone delivery for a total of three days. During these three days, the pellet 1 is not delivering hormones as it naturally would, but rather pauses the delivery in order to avoid an imbalance of hormones within the female body during the menstruation period. In other words, the portion of the quantity of the second medication 5 comprises no female hormones, and the second time interval is three days.

Subsequently, the method includes providing a dispensing time period, wherein the dispensing time period is governed by a predefined threshold for the quantity of the first medication 4 and the quantity of the second medication 5. In other words, the dispensing time period is dependent on the amount of medication stored within the pellet 1. Preferably, the time period is one year or 12 months. However, the time period may be increased or decreased, depending on the user's preference, the patient's health conditions, the type of the medication etc. The release algorithm continues the previously described hormonal release phase and the hormonal release pause for the entire duration of the pellet, or until it is otherwise removed. The duration of the pellet 1 or the dispensing time period may also be denoted as the pellet life-cycle. In other words, the pellet life-cycle is variable in duration, as it is able to perform the release algorithm for a life-cycle of three months, four months, six months or a year.

Continuing with the previous example, or in reference to FIG. 3, after completing a full cycle of twenty seven days of hormonal release and a three day hormonal pause, the release algorithm sets back to the hormonal release phase, wherein the pellet releases hormones at a regular rate for twenty seven consecutive days. More specifically, once the new cycle begins, the hormone pellets continue with the regular release for twenty seven consecutive days until the hormonal pause phase is reached at the conclusion of those twenty seven days. Subsequently, the release algorithm once again pauses the hormone release for three consecutive days, during the menstrual period of a woman. In other words, the present invention periodically activates the medication release mechanism 6 until the end of the dispensing time period. Thus, concluding another cycle from the release algorithm. That rhythm or the dispensing time period could last an entire year, which makes the present invention a comprehensive method to deliver hormones to women after menopause.

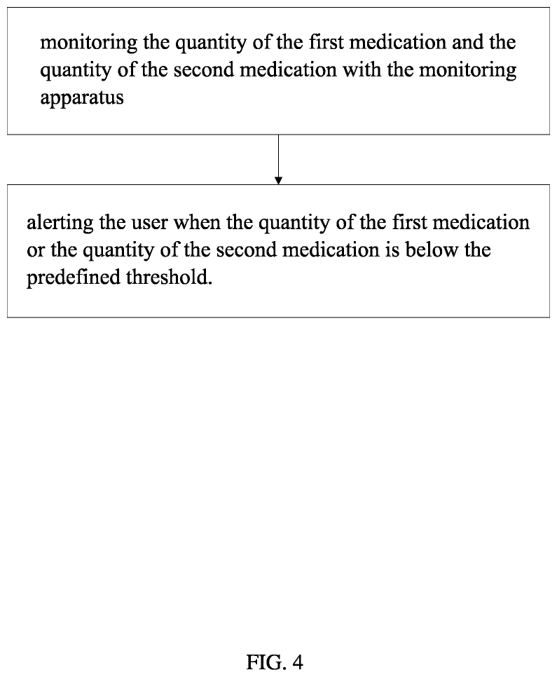

In reference to FIG. 1, the present invention may comprise a monitoring apparatus 7 for determining the amount of medication left in the pellet 1. To that end, and in reference to FIG. 4, the present invention comprises a method of execution for the monitoring apparatus 7 that comprises the following steps. Preferably, the method comprises monitoring the quantity of the first medication 4 and the quantity of the second medication 5 with the monitoring apparatus 7. The monitory apparatus 7 may include an external device that may be directly or indirectly connected to the pellet 1. Examples of such monitoring apparatus 7 includes, but are not limited to blood testing devices, scanning devices, chemical tracing devices, saliva testing devices etc. Subsequently, the user may be altered when the quantity of the first medication 4 or the quantity of the second medication 5 is below the predefined threshold. It should be noted that the monitoring apparatus 7 may comprise any other technology, components, or arrangement of components that are known to one of ordinary skill in the art, as long as the intents of the present invention are not altered.

In an alternate embodiment, the present invention is catered to a human male body. More specifically, the present invention may be used to treat men with prostate cancer through a treatment method known as ‘Bipolar Testosterone Replacement’. For this therapy, medical literature prescribes 14 days of high doses of testosterone and 14 days of placebo or varying testosterone/antiandrogenic drug. Thus, in this case, the first time interval may be approximately 14 days, and the second time interval may be approximately 14 days. In other words, the first time interval will comprise 14 days of releasing high testosterone (quantity of first medication) and the second time interval will comprise 14 days of releasing antiandrogenic drugs (quantity of second medication), making neuroreceptors more responsive to the upcoming hormones in the second cycle or the first medication.

It is an aim of the present invention to maintain correct and natural levels of hormones naturally produced by the human body cyclically following human body's nature. To that end, the first medication and the second medication are biodegradable. In other words, the hormone pellet comprises bioidentical hormones as the ones produced by the human body. Some of the bioidentical hormones that the hormone pellets comprise includes, but is not limited to testosterone, estradiol, estriol, and progesterone. Additionally, other components may also be included such as anastrozole, letrozole, or finasteride.

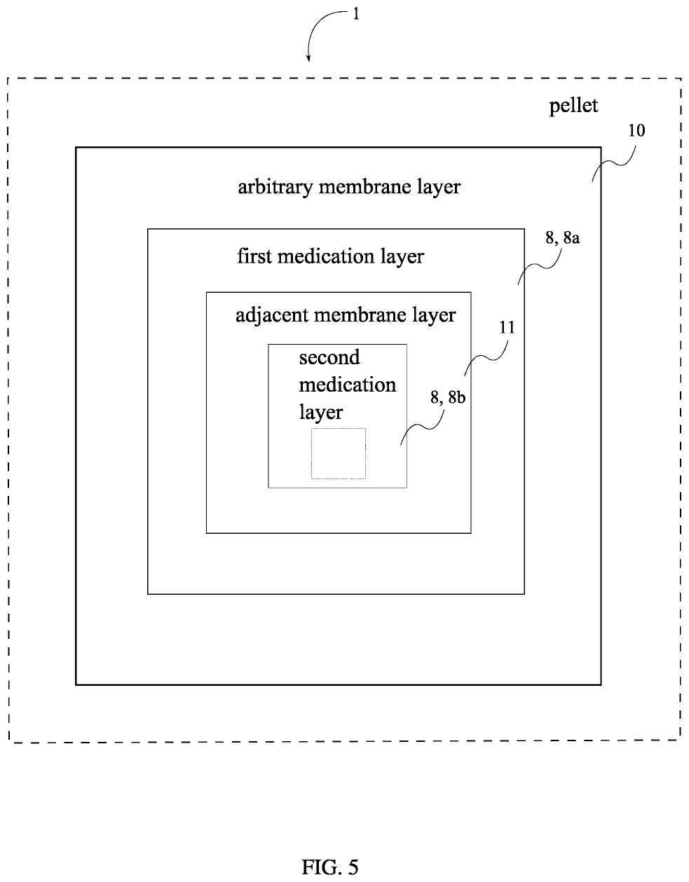

In reference to FIG. 5, a more specific embodiment of the present invention follows. Accordingly, the present invention comprises a pellet 1, and the pellet 1 comprises a plurality of medication layers 8 and a plurality of membrane layers 9. In this embodiment, each of the plurality of medication layers 8 is sandwiched in between an arbitrary membrane layer 10 and an adjacent membrane layer 11, wherein the arbitrary membrane layer 10 and the adjacent membrane layer 11 are from the plurality of membrane layers 9. In other words, intermediary membrane layers may wrap around medication layers and the medication may be slowly released as the intermediary membrane dissolves. To that end, the plurality of medication layers 8 comprises a plurality of first medication layers 8a and a plurality of second medication layers 8b, wherein the plurality of first medication layers 8a and the plurality of second medication layers 8b are arranged in a predefined order.

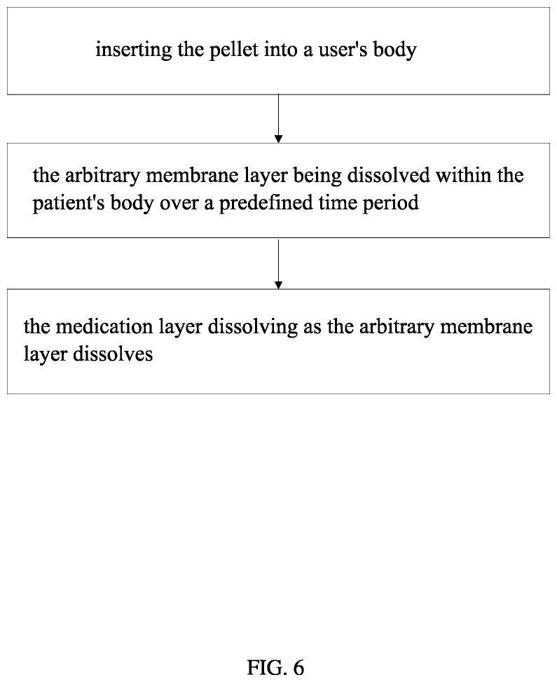

In order to accomplish the above mentioned functionality, the present invention follows a method of operation. As seen in FIG. 6, a first step of the method includes inserting the pellet into the patient. Preferably, a physician applies a local anesthetic, and performs a small incision located on the upper hip area. The pellet is then inserted, and standard medical tape is used to seal the cut, rather than stiches. However, any other method that is known to one of ordinary skill in the art may be employed. Following the insertion process, the arbitrary membrane layer 10 is dissolved within the patient's body over a predefined time period. The predefined time period depends on the patient and the medical condition that is intended to be treated using the pellet therapy. For example, the first medication layer 8a dissolves as the arbitrary membrane layer 10 dissolves, and the second medication layer 8b dissolves as the adjacent membrane layer 11 dissolves. In other words, as each membrane layer dissolves, the subsequent medicine layer gets dissolved into the user's body, and the process continues till all of the plurality of membrane layers 9 and the plurality of medication layers 8 dissolve. Preferably, the plurality of first medication layers 8a comprises biodegradable hormones and the plurality of second medication layers 8b comprises biodegradable and non-hormonal placebo inducing medication.

According to the preferred embodiment of the present invention, the plurality of first medication layers 8 is dissolved at a first constant rate, and the plurality of second medication layers 8b is dissolved at a second constant rate. This is because, the pellets are placed on the subcutaneous tissue to release small and regular doses of hormones into the body. The goal of the present invention is to maintain correct and natural levels of hormones naturally produced by the human body cyclically following human body's nature. One of the benefits of hormone pellet therapy is the small doses of hormones which are released into the body. Accordingly, the first constant rate and the second constant rate are chosen appropriate to the patient and the kind of medication used within the pellet 1. Thus, the present invention or pellet therapy that release hormones cyclically is a more reliable option, as the present invention releases a constant dose of hormones with intervals into the body, avoiding the negative effects of the high levels of hormones for an extended period of time and fast rate drop of hormones.

Any slight modification of the previously disclosed invention is to be considered still within the scope of the invention and should not be considered as a limiting factor for the present invention.

Any alternate embodiment that is derived from this concept and disclosure should also be considered within the scope of this invention, particularly since the female body may comprise certain variances within the natural cycles menstruation and associated biological processes.

Furthermore, the figures present within this document only serve as a visual aid and guide as to further illustrate the invention, and by no means limit the scope of the invention.

The invention has been explained in relation to its preferred embodiment, but it is to be understood that many other possible modifications and variations can be made without departing from the spirit and scope of the invention. Obvious changes, modifications and substitutions may be made by those skilled in the art to achieve the same purpose of the invention.

Although the invention has been explained in relation to its preferred embodiment, it is to be understood that many other possible modifications and variations can be made without departing from the spirit and scope of the invention as hereinafter claimed.

What is claimed is:

1. A system for treating a medical condition comprising:
a pellet;
the pellet comprising a pellet body, a plurality of release pathways, a quantity of a first medication, a quantity of a second medication, and a medication release mechanism;
the plurality of release pathways being integrated into the pellet body;
the quantity of the first medication being housed within the pellet body;
the quantity of the second medication being housed within the pellet body;
the medication release mechanism being housed within the pellet body;
the medication release mechanism being operatively coupled in between the plurality of release pathways, the quantity of the first medication and the quantity of the second medication, wherein the medication release mechanism dispenses a portion of the quantity of the first medication and a portion of the quantity of the second medication;
administering the pellet into a user's body;
dispensing the portion of the quantity of the first medication for a first time interval with the medication release mechanism;
dispensing the portion of the quantity of the second medication for a second time interval with the medication release mechanism;

2. The system of claim 1, comprising:
providing a dispensing time period, wherein the dispensing time period being governed by a predefined threshold for the quantity of the first medication and the quantity of the second medication;
periodically activating the medication release mechanism until the end of the dispensing time period.

3. The system of claim 2, comprising:
a monitoring apparatus;
monitoring the quantity of the first medication and the quantity of the second medication with the monitoring apparatus; and
alerting the user when the quantity of the first medication or the quantity of the second medication is below the predefined threshold.

4. The system of claim 2, wherein the dispensing time period being 12 months.

5. The system of claim 1, wherein the pellet being catered for a female human body:
the first time interval being 27 days; and
the second time interval being 3 days.

6. The system of claim 1, wherein administering the pellet into a patient's body comprises a subcutaneous injection method.

7. The system of claim 1, wherein the pellet being catered for a male human body:
the first time interval being approximately 14 days; and
the second time interval being approximately 14 days.

8. The system of claim 1, wherein the first medication and the second medication being biodegradable.

9. A system for treating a medical condition comprising:
a pellet;
the pellet comprising a plurality of medication layers, and a plurality of membrane layers;
each of the plurality of medication layers being sandwiched in between an arbitrary membrane layer and an adjacent membrane layer, wherein the arbitrary membrane layer and the adjacent membrane layer are from the plurality of membrane layers;
the plurality of medication layers comprising a plurality of first medication layers and a plurality of second medication layers, wherein the plurality of first medication layers and the plurality of second medication layers being arranged in a predefined order;
inserting the pellet into the patient;
the arbitrary membrane layer being dissolved within the patient's body over a predefined time period; and
the medication layer dissolving as the arbitrary membrane layer dissolves.

10. The system of claim 9, wherein the plurality of first medication layers comprising biodegradable hormones.

11. The system of claim 9, wherein the plurality of second medication layers comprising biodegradable and non-hormonal placebo inducing medication.

12. The system of claim 9, wherein the plurality of medication layers being catered to a female human body.

13. The system of claim 9, wherein the plurality of medication layers being catered to a male human body.

14. The system of claim 9, wherein the predefined time period being 12 months.

15. The system of claim 9, wherein administering the pellet into a patient's body comprises a subcutaneous injection method.

16. The system of claim 9, comprising:
the plurality of first medication layers being dissolved at a first constant rate; and
the plurality of second medication layers being dissolved at a second constant rate.
